(19) United States
(12) Patent Application Publication
(10) Pub. No.: US 2010/0210931 A1
(43) Pub. Date: Aug. 19, 2010

(54) METHOD FOR PERFORMING QUALITATIVE AND QUANTITATIVE ANALYSIS OF WOUNDS USING SPATIALLY STRUCTURED ILLUMINATION

(75) Inventors: David Cuccia, Costa Mesa, CA (US); Anthony J. Durkin, Irvine, CA (US); Joon S. You, Irvine, CA (US)

Correspondence Address: Law Offices of Daniel L. Dawes;Dawes Patent Law Group, 5200 Warner Blvd, Ste. 106, Huntington Beach, CA, 92649, US

(73) Assignee: Modulate Imaging Inc., Irvine, CA (US)

(21) Appl. No.: 12/418,515

(22) Filed: Apr. 3, 2009

Related U.S. Application Data
(60) Provisional application No. 61/042,479, filed on Apr. 4, 2008.

(51) Int. Cl. A61B 5/1455; A61B 6/00

(52) U.S. Cl. 600/328; 600/476

(57) ABSTRACT

A method of noncontact imaging for performing qualitative and quantitative analysis of wounds includes the step of performing structured illumination of surface and subsurface tissue by both diffuse optical tomography and rapid, wide-field quantitative mapping of tissue optical properties within a single measurement platform. Structured illumination of a skin flap is performed to monitor a burn wound, a diabetic ulcer, a decubitis ulcer, a peripheral vascular disease, a skin graft, and/or tissue response to photomodulation. Quantitative imaging of optical properties is performed of superficial (0-5 mm depth) tissues in vivo. The step of quantitative imaging of optical properties of superficial (0-5 mm depth) tissues in vivo comprises pixel-by-pixel demodulating and diffusion-model fitting or model-based analysis of spatial frequency data to extract the local absorption and reduced scattering optical coefficients.

### RELATED APPLICATIONS

The present application is related to U.S. Provisional Patent Application Ser. No. 61/042,479, filed on Apr. 4, 2008, which is incorporated herein by reference and to which priority is claimed pursuant to 35 USC 119.

### BACKGROUND OF THE INVENTION

1. Field of the Invention

The invention relates to the field of apparatus and method for performing qualitative and quantitative analysis of tissue using spatially structured illumination for qualitative and quantitative analysis of wounds.

2. Description of the Prior Art

The management of chronic wounds refers to those non-healing or delayed-healing wounds typically of cutaneous injuries. In ordinary wounds, the sequential healing process occurs through an orderly and timely fashion and results in a restoration of anatomic and functional integrity of tissues. On the other hand, a chronic wound occurs when systemic or environmental factors cause the disruption of the normal controlled inflammatory response and results in delayed and poor wound healing process. Chronic wounds may take an extended period to achieve an apparent healing, but the wound recurs, because it is unable to sustain closure. Most of chronic wounds start as simple superficial skin lesions. Although not usually fatal, these chronic wounds severely affect patients' quality of life because of impaired mobility and substantial loss of productivity. An estimated 6.5 million chronic wounds occur in the United States each year and the incidence is expected to increase as the population ages. Annual medical costs and lost productivity due to chronic wounds are estimated at several billion dollars in the U.S. Contributing to these staggering costs are treatment regimens that are expensive and ineffective. Chronic wound management is generally aimed at eliminating trauma, reducing ischemia, and minimizing bacterial infections, while providing an ideal healing environment (i.e. early closure).

The current state of sub-optimal management of chronic wounds is in large part due to the lack of objective and quantitative tools for assessment and monitoring of physiologic abnormalities within the chronic wound Ischemia is one of the main underlying physiologic problems contributing to impaired wound healing in patients. Ischemia of wound tissue occurs primarily in patients with vascular disease, diabetes, and in immobilized patients, such as quadriplegics and bed-bound individuals, due to the chronic action of pressure. Prolonged ischemia can lead to death of the affected tissue. Ischemia is typically a result of compromised vascular systems with inadequate blood perfusion and tissue oxygenation. Impaired perfusion and reduced oxygen tension in wound bed can delay early healing process involving re-vascularization by slowing the production of collagen. Furthermore, compromised tissue perfusion and oxygenation prevents proper healing because it provides a growth medium for bacteria, increasing the probability of infection.

In order to provide optimal treatment for chronic wounds, ischemia is one of the factors that must be alleviated, as well as reducing trauma to the tissue and bacterial contamination. Therapeutic strategies exist for improving tissue oxygenation and subsequent healing; however the tools that currently exist for making informed wound management decisions are suboptimal. Thus, cost-effective and user-friendly diagnostic devices for quantitative assessment and monitoring of tissue oxygenation and perfusion will facilitate efficient management of chronic wounds.

Consider first the measurement of blood flow and tissue oxygenation in wounds. Use of instruments to assess etiology and status of chronic wounds is still in an embryonic state. Clinicians rely primarily on clinical features such as wound size, location, depth, and infection in order to make treatment decisions. However, a promising array of medical devices under investigation for wound assessment includes Doppler ultrasound, Doppler perfusion imaging, transcutaneous measurement of tissue oxygen and near-infrared spectroscopy Blood flow has been considered a primary indicator of hemodynamic status of tissue. Ultrasound Doppler is a common clinical tool used to measure blood flow in arterial circulation. However, this suffers from a number of major problems that have inhibited widespread acceptance as a standard method of wound assessment. Specifically, the probe requires contact with the surface, therefore it is highly sensitive to movement and difficult to calibrate. Generally, the information content is presented in terms of relative flux and does not provide quantities that can be used in objective assessment. Another technique for measuring blood flow is laser Doppler perfusion imaging (LDI). LDI is a noninvasive non-contact instrument developed in the late 1980s to investigate the skin microvasculature. Its advantage is that it renders a two-dimensional flow map of a specific tissue, which allows a clinician to visualize the spatial variation of perfusion. Laser Doppler can noninvasively monitor flow changes, but most systems measure the tissue surface only (i.e., penetration depth<500 μm).

There are a number of practical problems that limit the usefulness of the laser Doppler method. Foremost among these is that sensitivity to movement artifact results in a poor signal-to-noise ratio. In addition, the output signal blood flux is in arbitrary units, which limits its uses in providing quantitative measures of blood perfusion and oxygenation state. Measurement of blood flow alone does not provide adequate information about status of cutaneous wounds. This is particularly true for chronic wounds with a significant amount of arteriovenous shunting where blood flow bypasses the capillary bed because such shunting maintains blood flow but does not provide nutrient (I.e. O₂) to the capillary bed and tissues. A rather direct determination of oxygen tension at the skin can be accomplished by transcutaneous oxygen sensors (Tcp0₂). Tcp0₂ measures the partial-pressure oxygen driving oxygen molecules through the dermal and epidermal layers and a membrane covering the sensor. It works by heating the skin to dilate the capillaries (small blood vessels) and measuring the resultant changes in the partial pressure of oxygen. Thus it is a measurement of trends rather than absolute quantities. As a surface measurement, it is insensitive to p0₂ changes within underlying wound bed, which provides the nutrient to the healing process. It is thus susceptible to errors due to such factors as local edema, skin thickening, inflammation, and local O₂ variability, all of which are common to wounds.

Consider now diffuse optical spectroscopy. Recently there has been considerable research in the use of diffuse optical spectroscopy (DOS) as a means for real-time in-vivo measurement of both tissue oxygenation and blood volume. DOS is a technique that combines experimental measurements and model-based data analysis to measure the bulk absorption (μₐ) and scattering (μₛ′) properties of highly scattering media. DOS instruments typically use red and near-infrared (NIR) light, especially from 600 to 1000 nm, where light propagation in tissue is scattering dominated. Diffusive photons probe a large sample volume, providing macroscopically averaged absorption and scattering properties at depths up to a few centimeters. Measurements of tissue optical properties are assumed to contain tissue structural and functional information. In the 600-1000 nm spectral region, the dominant molecular absorbers in tissue are oxygenated (Hb-0₂) and reduced hemoglobin (Hb-R), water, and lipids. DOS measurements yield absolute values of total hemoglobin, deoxyhemoglobin, and oxyhemoglobin in milligrams per milliliter, in addition to tissue oxygen saturation in percent. This can be done in real-time mode, allowing direct comparison between different regions of skin and individuals. Total hemoglobin is calculated by adding hemoglobin and oxyhemoglobin, revealing changes in tissue blood volume and providing indirect information on blood flow and perfusion. The oxygenation index can be calculated as the difference of oxyhemoglobin and hemoglobin, detecting changes in oxygenation independent of changes in blood volume.

An apparatus and method for performing qualitative and quantitative analysis of tissue using spatially structured illumination was disclosed in U.S. Pat. No. 6,958,815 and U.S. patent application Ser. No. 11/336,065, entitled “Method and Apparatus for Spatially Modulated Fluorescence Imaging and Tomography”, both of which are incorporated herein by reference. Several companies are now marketing devices that can be used to monitor skin flaps. These companies include Spectros Inc. T-Scan. and Vioptix. Hypermed is developing a hyperspectral imager for monitoring diabetic ulcers. However all of these approaches are small volume, fiber based nonimaging approaches.

In U.S. Pat. No. 6,958,815 we presented a disclosure involving wide field, broadband, spatially modulated illumination of turbid media. This approach has potential for simultaneous surface and subsurface mapping of media structure, function and composition. This method can be applied with no contact to the medium over a large area, and could be used in a variety of applications that require wide-field image characterization. The approach described in U.S. Pat. No. 6,958,815 is further refined and a fluorescence imaging capability is described in U.S. patent application Ser. No. 11/336,065, “Method and apparatus for Spatially Modulated Fluorescence Imaging and Tomography”, referenced above.

Use of instruments to assess etiology and status of chronic wounds is still in an embryonic state. Clinicians rely primarily on clinical features such as wound size, location, depth, and infection in order to make treatment decisions. However, a promising array of medical devices under investigation for wound assessment includes Doppler ultrasound, Doppler perfusion imaging, transcutaneous measurement of tissue oxygen and near-infrared spectroscopy. Blood flow has been considered a primary indicator of hemodynamic status of tissue. Ultrasound Doppler is a common clinical tool used to measure blood flow in arterial circulation.

However, this suffers from a number of major problems that have inhibited widespread acceptance as a standard method of wound assessment. Specifically, the probe requires contact with the surface, therefore it is highly sensitive to movement and difficult to calibrate. Generally, the information content is presented in terms of relative flux and does not provide quantities that can be used in objective assessment.

Another technique for measuring blood flow is laser Doppler perfusion imaging (LDI). LDI is a noninvasive non-contact instrument developed in the late 1980s to investigate the skin microvasculature. Its advantage is that it renders a two-dimensional flow map of a specific tissue, which allows a clinician to visualize the spatial variation of perfusion. Laser Doppler can noninvasively monitor flow changes, but most systems measure the tissue surface only (i.e., penetration depth<500 μm). There are a number of practical problems that limit the usefulness of the laser Doppler method. Foremost among these is that sensitivity to movement artifact results in a poor signal-to-noise ratio. In addition, the output signal blood flux is in arbitrary units, which limits its uses in providing quantitative measures of blood perfusion and oxygenation state.

Measurement of blood flow alone does not provide adequate information about status of cutaneous wounds. This is particularly true for chronic wounds with a significant amount of arteriovenous shunting where blood flow bypasses the capillary bed because such shunting maintains blood flow but does not provide nutrient (i.e. O₂) to the capillary bed and tissues. A rather direct determination of oxygen tension at the skin can be accomplished by transcutaneous oxygen sensors (Tcp0₂). Tcp0₂ measures the partial-pressure oxygen driving oxygen molecules through the dermal and epidermal layers and a membrane covering the sensor. It works by heating the skin to dilate the capillaries (small blood vessels) and measuring the resultant changes in the partial pressure of oxygen. Thus it is a measurement of trends rather than absolute quantities. As a surface measurement, it is insensitive to pO₂ changes within underlying wound bed, which provides the nutrient to the healing process. It is thus susceptible to errors due to such factors as local edema, skin thickening, inflammation, and local O₂ variability, all of which are common to wounds.

One common drawback to afore-mentioned techniques is the fact that they all rely on indirect measurements of tissue health status. What is needed is some kind of a more direct indication of tissue health or metabolic status of tissues at a cellular level.

In order for DOS technology to become widely accepted for assessment and monitoring of wounds, it is critical that the new technique overcomes key clinical challenges. Some of these challenges are inherent in measurement methodologies. For example, any contact probe will suffer from tissue structure heterogeneities, edema, user variability, site variability and so forth. Thus, a non-contact imaging modality is preferred for practical use in the clinics. Imaging mode of DOS technologies have been developed and successfully applied to breast and brain tissue measurements but they are too expensive and impractical for imaging superficial wounds.

### BRIEF SUMMARY OF THE INVENTION

In the illustrated embodiment of the invention we describe a method, based on modulated imaging or structured illumination for surface and subsurface quantization of wound tissue or superficial wounds. Hereinafter wherever the term, “structured illumination” is used, it is to be understood as including modulated imaging as one modality. We demonstrate this method using a rat skin flap model. Applications include skin flap monitoring, burn wound management, diabetic ulcers, decubitis ulcers, peripheral vascular disease monitoring. A more direct indication of tissue health or metabolic status of tissues at a cellular level can be made by measuring local concentrations and oxygen saturation of hemoglobin in capillary bed. A potential technique for real-time in-vivo measurement of both blood volume and cellular metabolism in skin tissue is diffuse optical spectroscopy via structured illumination.

A noncontact imaging modality is preferred for practical use in the clinics. Imaging mode of DOS technologies have been developed and successfully applied to breast and brain tissue measurements but they are too expensive and impractical for imaging superficial wounds. Structured illumination is a unique imaging modality that is based on the DOS principles and is ideal for imaging subsurface tissues. Structured illumination is a novel noncontact optical imaging technology under development at the Beckman Laser Institute. Compared to other imaging approaches, structured illumination has the unique capability of performing both diffuse optical tomography and rapid, wide-field quantitative mapping of tissue optical properties within a single measurement platform. We demonstrate this method using a rat skin flap model. Applications include skin flap monitoring, burn wound management, diabetic ulcers. decubitis ulcers, peripheral vascular disease monitoring.

Structured illumination shows great promise for quantitative imaging of optical properties of superficial (0-5 mm depth) tissues in vivo, Pixel-by-pixel demodulation and diffusion-model fitting or model based analysis of spatial frequency data is performed to extract the local absorption and reduced scattering optical coefficients. When combined with multispectral imaging, absorption spectra at each pixel can be separately analyzed to yield spatial maps of local oxy and deoxy hemoglobin concentration, and water concentration. Total hemoglobin (THb) and oxygen saturation (stO₂) maps can then be calculated as THb=HHb+O₂Hb and stO₂=O₂Hb/[HHb+O₂Hb]*100, respectively.

Impaired perfusion and oxygenation are one of the most frequent causes of healing failure in chronic wounds such peripheral vascular disease, diabetic ulcers and pressure ulcers. These ulcers always require immediate intervention to prevent progression to a more complicated and potentially morbid wound. Thus, development of noninvasive technologies for evaluation of tissue oxygenation and perfusion of the wound is essential for optimizing therapeutic treatments of chronic wounds. We have developed a means for quantitatively monitoring superficial wounds.

More particularly, the illustrated embodiment of the invention includes a method of noncontact imaging for performing qualitative and quantitative analysis of wounds comprising the step of performing structured illumination of surface and subsurface tissue by both diffuse optical tomography and rapid, wide-field quantitative mapping of tissue optical properties within a single measurement platform.

The step of performing structured illumination of surface and subsurface tissue comprises performing structured illumination to monitor a skin flap, a burn wound, a diabetic ulcer, a decubitis ulcer, a peripheral vascular disease, a skin graft, a bruise, and/or tissue response to photomodulation.

The step of performing structured illumination of surface and subsurface tissue comprises quantitative imaging of optical properties of superficial (0-5 mm depth) tissues in vivo.

The step of quantitative imaging of optical properties of superficial (0-5 mm depth) tissues in vivo comprises pixel-by-pixel demodulating and diffusion-model fitting or model based analysis of spatial frequency data to extract the local absorption and reduced scattering optical coefficients.

The step of performing structured illumination of surface and subsurface tissue further comprises multispectral imaging to separately analyze absorption spectra at each pixel to yield spatial maps of local oxy and deoxy hemoglobin concentration, and water concentration and to calculate total hemoglobin (THb) and oxygen saturation (SₜO₂)(maps can then be calculated as THb=HHb+O₂Hb and SₜO₂=O₂Hb/[HHb+O₂Hb]*100, respectively.

Another embodiment of the invention includes a method of imaging comprising the step of structured illumination of a cutaneous wound to spatially resolve quantitative maps of tissue hemoglobin, oxygenation and/or hydration in the wound.

The step of structured illumination of a cutaneous wound to spatially resolve quantitative maps of tissue hemoglobin content and oxygen saturation in the wound comprises structured illumination at various spatial frequencies can be processed to visualize depth-sectioned subsurface features in terms of scattering and absorption.

The method further comprises the step of mapping the absorption coefficient at each wavelength in a predetermined spectral segment to perform quantitative spectroscopy of tissue.

The step of mapping the absorption coefficient at each wavelength in a predetermined spectral segment to perform quantitative spectroscopy of tissue comprises mapping extinction coefficients of the tissue chromophores, including Hb0₂, Hb, and H₂0 and other endogenous chromophores (e.g. melanin, lipids (fat), other hemoglobins and heme breakdown products.

The step of mapping extinction coefficients of the tissue chromophores, including Hb0₂, Hb, and H₂0 comprises mapping concentration of oxy and deoxy-hemoglobin over the vein regions by calculating the tissue-level oxygen saturation (Sₜ0₂=Hb/[Hb+Hb0₂]), and highlighting the effect of tissue oxygen extraction.

The step of mapping extinction coefficients of the tissue chromophores, including Hb0₂, Hb, and H₂0 comprises mapping a sum of Hb and Hb0₂ to yield HbT, the total hemoglobin concentration to obtain a direct, absolute measure of blood volume in tissue.

The step of mapping extinction coefficients of the tissue chromophores, including Hb0₂, Hb, and H₂0 comprises mapping H₂O at or near the water peak of 970 nm to provide a direct mapping of tissue water concentration.

The step of structured illumination of a cutaneous wound to spatially resolve quantitative maps of tissue hemoglobin, oxygenation and/or hydration in the wound comprises structured illumination to spatially resolve quantitative maps of tissue hemoglobin content and oxygen saturation in chronic wounds undergoing ischemia.

The step of structured illumination of a cutaneous wound to spatially resolve quantitative maps of tissue hemoglobin, oxygenation and/or hydration in the wound further comprises depth-sectioned imaging to enhance sensitivity to the physiologic changes in superficial wounds.

The step of structured illumination of a cutaneous wound to spatially resolve quantitative maps of tissue hemoglobin, oxygenation and/or hydration in the wound further comprises imaging using 690, 750, 830 and 980 nm light in a modulated pattern.

The step of structured illumination of a cutaneous wound to spatially resolve quantitative maps of tissue hemoglobin, oxygenation and/or hydration in the wound further comprises structured illumination of a cutaneous wound with online data processing to enable immediate feedback on flap health status, to reduce sensitivity to motion artifacts, to and create an ability to track small, subtle changes that may occur during surgery.

The step of structured illumination of a cutaneous wound to spatially resolve quantitative maps of tissue hemoglobin, oxygenation and/or hydration in the wound comprises identifying perfusion changes at tissue depths of 1 cm or less.

The step of structured illumination of a cutaneous wound to spatially resolve quantitative maps of tissue hemoglobin, oxygenation and/or hydration in the wound comprises performing the structured illumination with no more than two spatial frequencies to allow for rapid online data processing of an image.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1a is a graph of the depth-dependence of spatially-modulated wave in tissue shown at a series of increasing tissue depths.

FIG. 1b is a graph of the penetration depth in mm of the illumination as a function of frequency in mm⁻¹.

FIG. 2a is a graph of φ_{AC} as a function of depth in mm illustrating depth-sectioning and FIG. 2b is a graph of φ_{AC} at z=0 in relative units as a function of spatial frequency in mm⁻¹ illustrating optical property sensitivity of spatially-modulated illumination.

FIG. 4 is a two dimensional map of a homogenous phantom of the absorption μₐ and reduced scattering μₛ′ coefficients on the left with corresponding pixel histograms of the same on the right.

FIG. 5a is a diagram of a heterogeneous phantom and FIG. 5b is a reconstructed absorption tomograph of the tissue simulating phantom of FIG. 5a using the spatial frequency-dependent depth penetration of spatially modulated illumination.

FIG. 6a is an image of a region of interest (ROI) in a brain. FIG. 6c shows spatially-averaged modulation data and fitting results for three sample wavelengths. FIG. 6b is a graph of the mean absorption (μₐ) and in FIG. 6d scattering (μₛ′) vs. wavelength with detailed results at sample wavelengths listed below FIG. 6d.

FIG. 7a is a graph of quantitative absorption and FIG. 7b is a graph of scattering maps at 650 nm over a 3.8×4.9 mm field of view. FIGS. 7c and 7d are pixel histograms corresponding to the images of FIGS. 7a and 7b showing statistical distribution of recovered image values.

FIG. 8a at the top is a quantitative map of oxy-hemoglobin (Hb0₂), and at the bottom of deoxy-hemoglobin (Hb) and in FIG. 8d of water (H₂0) concentration maps over 3.8×4.9 mm field of view. FIG. 8b is a quantitative map of tissue O₂ saturation (Sₜ0₂), and total hemoglobin (HbT) maps, calculated from Hb and Hb0₂.

FIG. 9 includes three graphs of quantitative structured illumination data of the skin flap model 48 hrs post surgery, showing from left to right the diffuse reflectance, the absorption coefficient and the scattering coefficient as a function of wavelength. Measurements were made over a spectral range of 650 to 970 nm using a broadband quartz-tungsten-halogen light source, combined with a liquid crystal tunable filter. Four spatial frequencies were acquired, from 0 mm⁻¹ to 0.32 mm⁻¹.

FIGS. 10a-10d are photographs of the clinical appearance of the flaps during arterial and venous occlusion at time=2 min (FIG. 10a), arterial and venous complete occlusion at time=60 min (FIG. 10b), selective venous occlusion at time=2 min (FIG. 10c), and selective venous occlusion at time=30 min (FIG. 10d).

FIG. 11 show maps of the tissue Chromophore measurements at 2 minutes after either combined Arterial and Venous occlusion or Selective 100% Venous Occlusion. The control flap is shown on the right and the experimental flap on left. The graphs shown the control flap in the lower curve and the experimental flap in the upper curve.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

Impaired perfusion and oxygenation are one of the most frequent causes of healing failure in chronic wounds such peripheral vascular disease, diabetic ulcers and pressure ulcers. These ulcers always require immediate intervention to prevent progression to a more complicated and potentially morbid wound. Thus, development of noninvasive technologies for evaluation of tissue oxygenation and perfusion of the wound is essential for optimizing therapeutic treatments of chronic wounds. One promising technology for measuring tissue oxygenation in-vivo is diffuse optical spectroscopy (DOS) and imaging. DOS is a quantitative near-infrared (NIR) spectroscopy technique that can determine absolute concentrations of chromophores such as oxy/deoxy hemoglobin, fat and water and other endogenous chromophores (e.g. melanin, lipids (fat), other hemoglobins and heme breakdown products. These quantities may provide the simple objective measures for diagnosis and assessment of chronic wounds.

One object of the illustrated embodiment is to employ a new imaging method, known as structured illumination, to spatially resolve quantitative maps of tissue hemoglobin content and oxygen saturation in an animal wound model and therefore for use in humans as well. The structured illumination instrument uses patterned illumination to non-invasively obtain subsurface images of biological tissues. This non-contact approach enables rapid quantitative determination of the optical properties of the biological tissues over a wide field-of-view. When combined with multi-spectral imaging, the optical properties at several wavelengths provide quantitative measures within tissues to determine the in-vivo concentrations of chromophores, namely, oxy- and deoxy-hemoglobin and other endogenous chromophores (e.g. melanin, lipids (fat), other hemoglobins and heme breakdown products.

Furthermore, images at various spatial frequencies can be processed to visualize depth sectioned subsurface features in terms of scattering and absorption. Furthermore, images at various spatial frequencies can be processed to visualize depth-sectioned subsurface features in terms of scattering and absorption. Our hypothesis is that the NIR-based or even visible-light structured illumination instrument can effectively work as a tissue oxygenation imager or an “Oximager” for quantitative assessment of hemoglobin content and oxygenation within ischemic chronic wounds of superficial tissues.

The illustrated embodiment of the invention is intended to answer the following questions:
a. Can SI techniques be used to assess tissue oxygenation and perfusion status?
b. Can depth-sectioning capability of SI techniques be used to enhance its sensitivity to the tissue oxygenation changes?
c. What are the measurement parameters optimal for detecting physiologic changes?

To address these questions, we conducted a structured illumination study of superficial wounds using an animal skin flap model. The skin flap model can be easily implemented to establish controlled ischemia and re-perfusion of the wounds. This allows us to methodically evaluate the ability of structured illumination to deduce spatially resolved maps of tissue hemoglobin, oxygenation and/or hydration. In addition, the flap model provides us with an in-vivo means to evaluate that depth sectioning capabilities of structured illumination.

As an example, in the illustrated embodiment we implemented ischemic skin flaps in rats to simulate chronic wounds with compromised tissue oxygenation and perfusion. We acquired full range of multi-spectral, multi-spatial-frequency images of skin flaps before and after surgery We processed and optimized images for two and three dimensional mapping of hemoglobin concentrations, oxygen saturation, and water content in superficial wound. We first determined optimal spatial frequencies for imaging in-vivo tissue oxygenation, which includes 650 nm. This information was used to build a dedicated prototype imaging oximeter system for evaluating clinical wounds.

Structured illumination is a unique imaging modality that is based on the DOS principles and is ideal for imaging subsurface tissues. Consider first the principles of structured illumination. Structured illumination (SI) is a novel non-contact optical imaging technology under development at the Beckman Laser Institute, University of California, Irvine. Compared to other imaging approaches, SI has the unique capability of performing both diffuse optical tomography and rapid, wide-field quantitative mapping of tissue optical properties within a single measurement platform. While compatible with time-modulation methods, SI alternatively uses spatially-modulated illumination for imaging of tissue constituents. Periodic illumination patterns of various spatial frequencies are projected over a large area of a sample. The reflected image is modified from the illumination pattern due to the turbidity of the sample. Typically, sine-wave illumination patterns are used. The demodulation of these spatially-modulated waves characterizes the modulation transfer function (MTF) of the material, and embodies the sample's structural and optical property information. The spatial-frequency dependence of sample reflectance encodes both depth and optical property information.

Introducing a spatially-modulated source, Eq(2), into the steady state diffusion equation, Eq(1):

∇²φ−k²φ=S (1)

S=S_{0[}1+M sin(2πfₓx)] (2)

Where

k=\sqrt{3μₐ(μₐ+μₛ′)}=μ_{eff} (3)

and where 1/μ_{eff} is the effective penetration depth of the illumination, gives results:

∂_{z}²φ_{AC}−(k²+(2πfₓ)²)φ_{AC}=S₀ (4)

μ′_{eff}²=3μₐ(μₐ+μ′ₛ)+(2πfₓ)² (5)

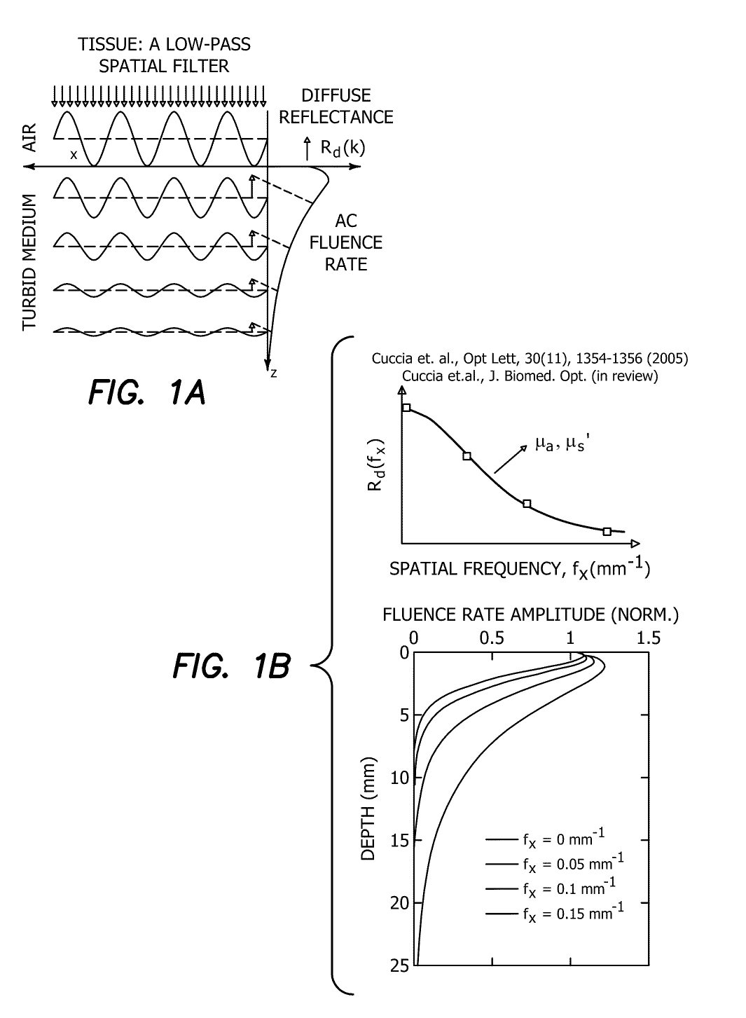

Here, φ is the internal fluence, S the illumination source, M the modulation depth of the illumination, and fₓ the spatial frequency of illumination, and φ_{AC} refers to the harmonically varying component of the fluence. The spatially-modulated wave propagates in turbid media as that from planar illumination source Sₒ would, except that the penetration depth, 1/μ_{eff}, depends on the spatial frequency of illumination, illustrated in FIGS. 1a and 1b.

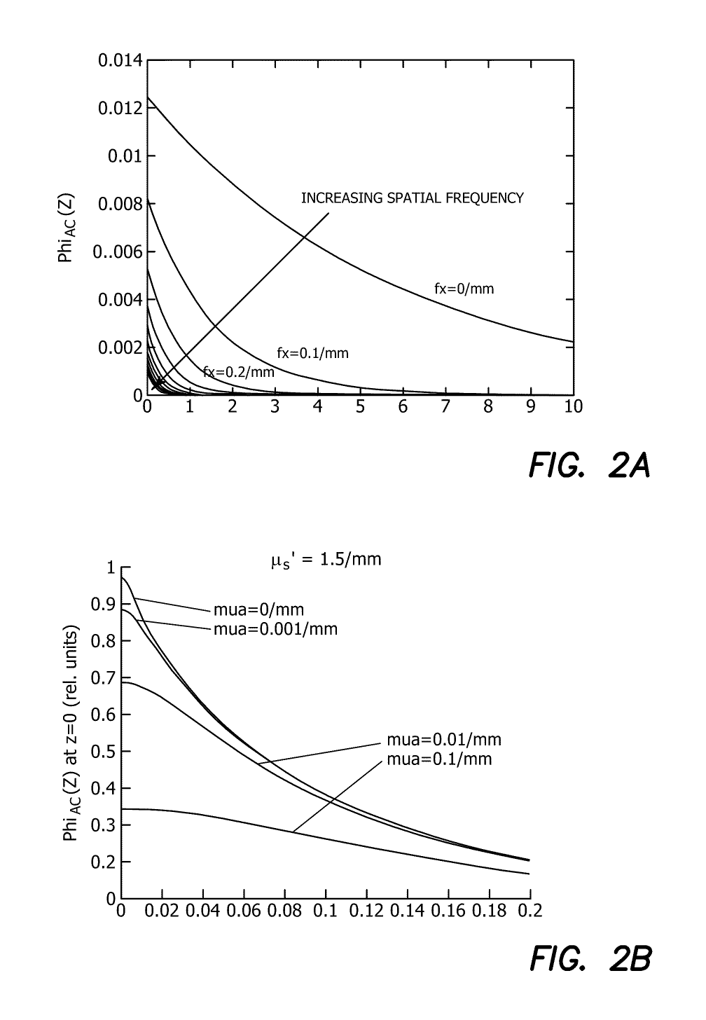

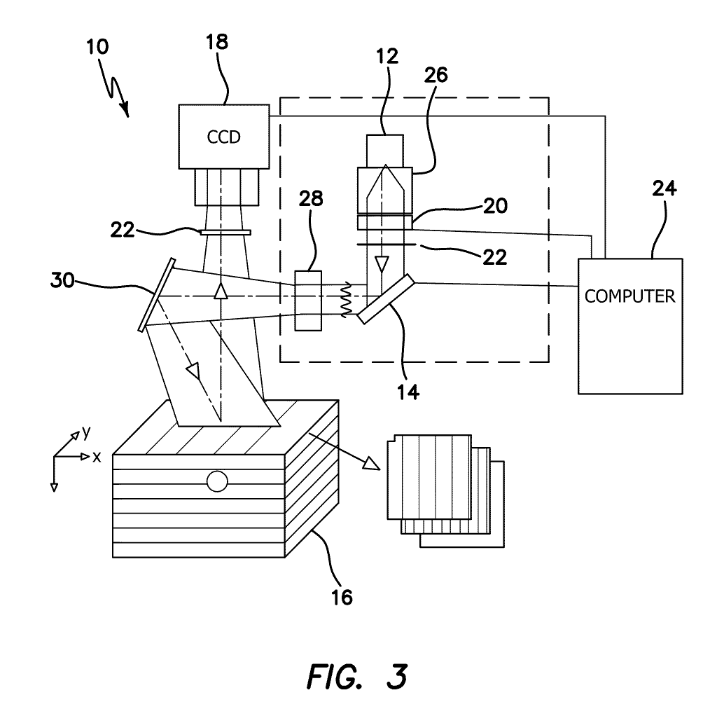

There are two major implications of Equations 4 and 5. First, varying the spatial frequency of the illumination pattern allows one to control the depth sensitivity of detection inside the turbid medium as illustrated in FIG. 2a. Second, by analyzing the frequency dependent reflectance, one can quantitatively sample the optical properties of the medium. Simulated frequency responses for varying optical properties, shown in FIG. 2b, demonstrate the potential for determination of optical properties. This is analogous to the frequency-domain photon migration (FDPM) technique, a variant of diffuse optical spectroscopy, where the temporal frequency of the photon density waves is related to the spatial frequency through the speed of photon density wave propagation in the medium of interest.

In practice, the illumination is in the form cos(2πfₓ+φ)+½, containing a DC component to allow for modulation from 0 to 1. In order to view the reflectance due to the AC and DC components separately, a standard technique in signal processing is employed. This requires illuminating the sample three times at the same spatial frequency, with phase offsets of 0, 120 and 240 degrees. An image of the AC modulated reflectance can be calculated using Eq (5),

$$AC = \frac{\sqrt{3}}{2} \sqrt{{( A - B )}^{2} + {( B - C )}^{2} + {( C - A )}^{2}} \qquad ( 5 )$$

where A, B, and C represent the reflectance images with shifted spatial phases. This has been recently employed for use in confocal microscopy.

Turn now and consider an example of a structured illumination instrument 10.

A schematic diagram of the structured illumination instrument 10 is depicted in FIG. 3. The light source 12 is a halogen lamp or laser whose beam, focused by a condenser 26 or other optics, is expanded to match the digital micromirror device 14. The digital micromirror device 14 is comprised of 1024×768 binary mirrors, based on the DLp™ technology developed by Texas Instruments, and is used to control the light pattern projected on the tissue 16 using a projector lens 28 and mirror 30 or other optics. The image reflected from tissue 16 is then recorded by a digital CCD camera 18, which includes for example a 512×512 imaging array. Each pixel acts similarly to an avalanche photodiode, simultaneously allowing very high sensitivity and dynamic range at fast readout rates (up to 10 MHz). A filter wheel 20 is used to select a discrete number of wavelengths. Linear polarizers 22 are introduced into the source and detection light paths to measure both parallel and perpendicular polarizations. The digital micromirror device 14, CCD camera 18 and filter wheel 20 are synchronized by a computer 24, enabling fast acquisition of a series of patterns with various spatial frequencies. The specular reflection is carefully avoided by illuminating at a small angle to the normal direction, and by using crossed linear polarizers 22. Interference filters (not shown) allow for narrow wavelength band selection. A spectralon reflectance standard was used to calibrate the measured intensity, and to correct for spatial nonuniformity in both the illumination and imaging systems.

The first set of experiments imaged siloxane phantoms that were designed to be homogeneous. The known ‘bulk’ optical properties at 640 nm were: μₐ=0.00736 mm⁻¹, μₛ′=0.901 mm⁻¹, as measured by large source-detector separation FDPM. Eleven, 3-image sets were acquired over a 5×5 cm² surface, with spatial frequencies ranging from 0 mm⁻¹ to 0.6 mm⁻¹. Modulation images at each frequency were obtained as previously described. The resulting 11 images provide a quantitative ‘frequency-response’, or modulation transfer function (MTF) of the diffuse reflectance of the turbid phantom. Moreover, this MTF is available at each pixel. Diffuse reflectance vs. frequency can be predicted analytically by taking a spatial Fourier transform of a spatially-resolved reflectance model. This enables phantom-based calibration and least squares regression to obtain the absolute optical properties of the sample. Here, phantom calibration accounts for both the lamp intensity and MTF of the imaging optics.

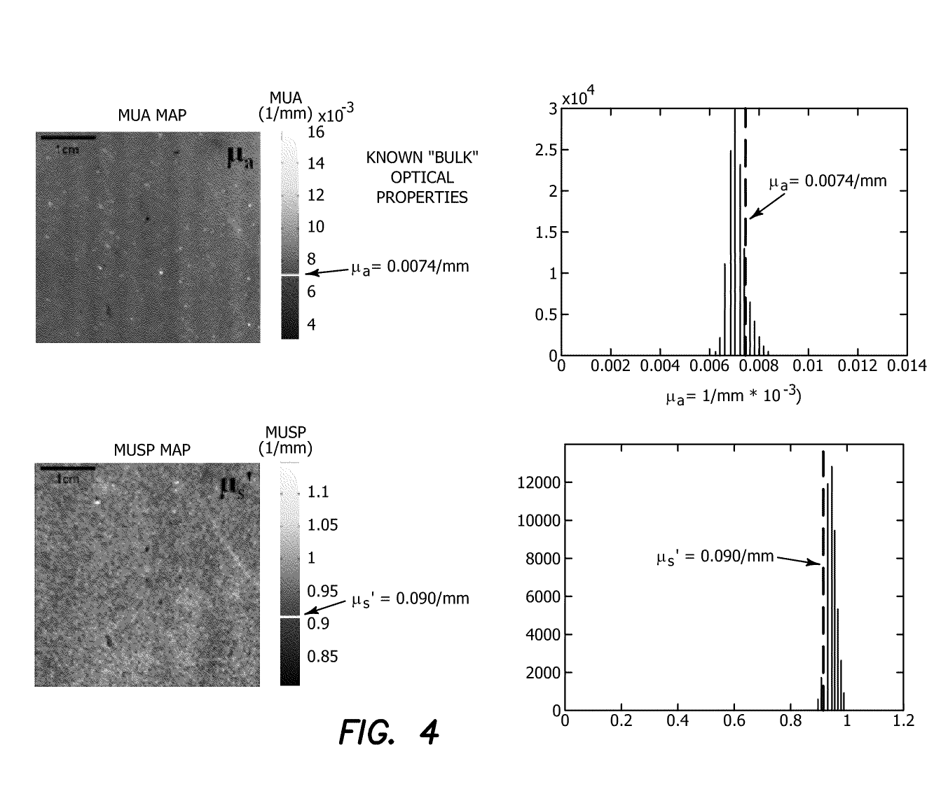

Because the AC amplitude is determined at each pixel, it is possible to do a pixel-by-pixel frequency fit. This was performed over the 5×5 cm² area (approx, 500×500 pixels). Maps of the recovered absorption and scattering properties are shown in FIG. 4. To the right of each map is a histogram of pixel values with a black dotted line indicating the known bulk values of μₐ=0.00736 mm⁻¹, μₛ′=0.901 mm⁻¹. The recovered properties are in very good agreement to the known bulk properties, with the bulk properties falling well within the corresponding histograms. These result agree very well with the known bulk properties, which were determined from large source˜detector separation FDPM measurements.

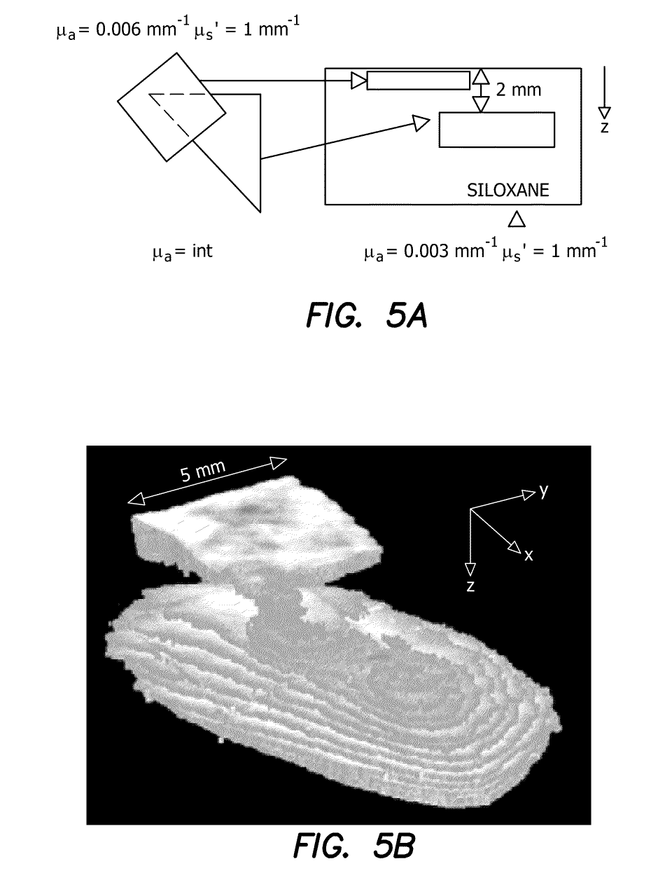

Consider now tomographic imaging with structured illumination of a heterogeneous phantom. Shown in FIGS. 5a and 5b is a diagram of a breast-like tissue-simulating phantom modified to accommodate two heterogeneities. A siloxane block containing Ti0₂ (μₐ=0.003 mm⁻¹, μₛ′=1 mm⁻¹ at 640 nm) was modified to accommodate two heterogeneities. The first one, an absorbing mask 32 (triangular in shape) was placed 2 mm inside the sample. The second heterogeneity was a scattering and absorbing element 34 (square in shape) placed at the surface of the siloxane block (thickness=0.5 mm, μₐ=0.006 mm⁻¹, μₛ′=1 mm⁻¹). A total of 126 images at 42 spatial frequencies were acquired, ranging from 0 to). 63 mm⁻¹. While the system was not optimized for speed, actual image acquisition time was approximately 24 seconds.

In FIG. 5b we show a three dimensional tomographic reconstruction of the structured illumination data set. The depth scale is marked from a priori knowledge of the phantom dimensions. The two objects are clearly resolved, with resolution degrading as depth into the sample increases. Quantitative reconstruction methods currently under development are expected to improve this resolution, aided by the robust measure of the sample's average optical properties. The initial data demonstrates that structured illumination can simultaneously accommodate the measurement of the optical properties over a wide field-of-view in addition to a fast and economical procedure to achieve depth sectioning in turbid media.

Proof-of-principle functional measurements were performed on an in-vivo rodent model. The skull of the anesthetized animal was thinned to allow direct imaging of the cortex (somatosensory region). Spatial modulation data were acquired at 8 evenly-spaced frequencies between 0 and 0.13 mm⁻¹ over a 5×7 mm field-of-view. This was performed at 10 nm intervals over the entire range between 650 and 990 nm using a 10 nm bandwidth liquid-crystal tunable filter camera (Nuance, CRI). Depending on the wavelength, acquisition time for all frequencies varied between 3.8 and 120 seconds for this prototype system, yielding a total measurement time of approximately 5 minutes. In an optimized imaging system with 4 wavelengths and 2 spatial frequencies, we believe total acquisition time could be reduced to approximately 1 second or less, resulting in frame rates>1 Hz.

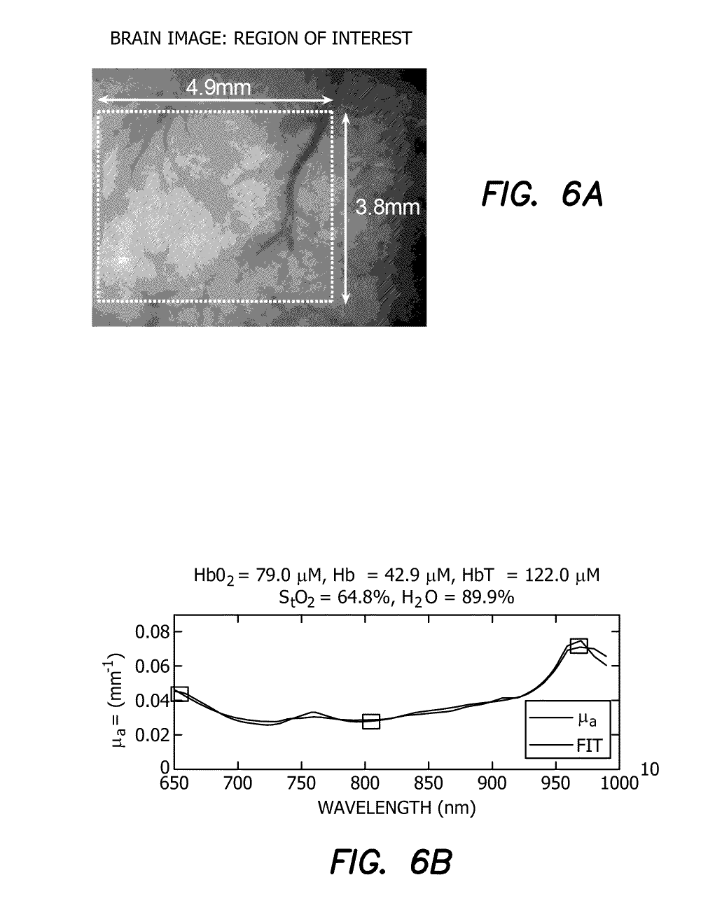

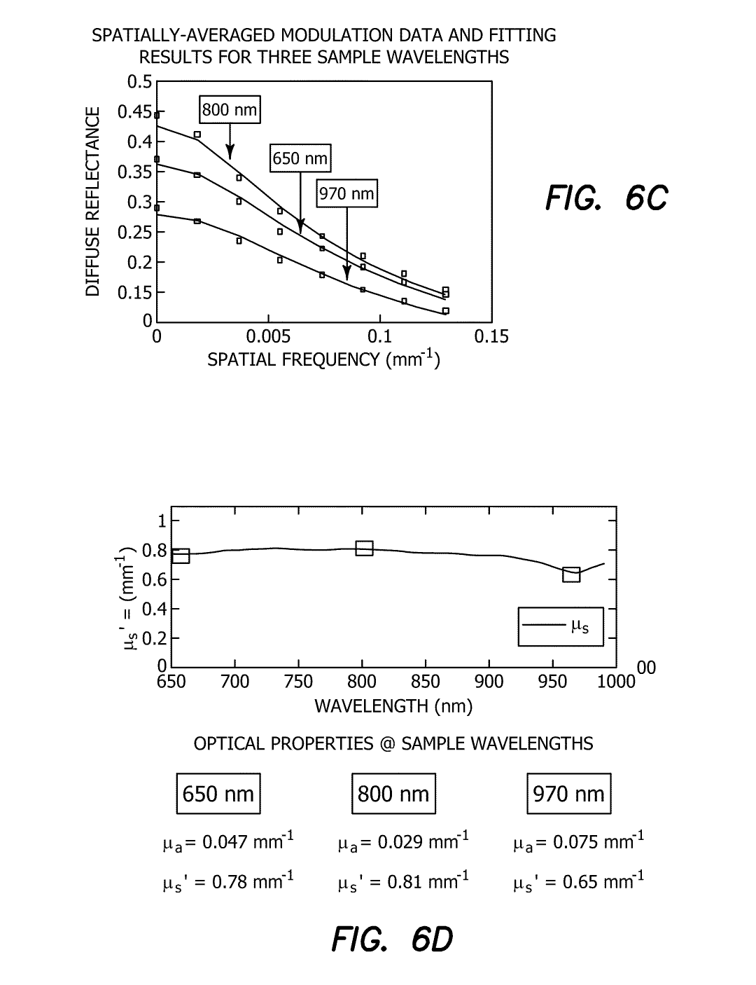

In FIG. 6a we show a grayscale image of the cortical region. A dotted-line box in the figure denotes the region-of-interest (ROI) used for analysis. This region was selected for its uniform illumination and the absence of cerebral bruising. FIG. 6c shows the sample frequency modulation measurements at selected wavelengths of 650, 800, and 970 nm. Here, the squares are average modulation data over the entire ROI, and the lines are the resulting non-linear least squares fits using a diffusion model for light transport. In FIG. 6b we show the spatially-averaged, quantitative absorption (μₐ) and in FIG. 6d the reduced scattering (μₛ′) measurements versus wavelength. Note the distinct spectral features in absorption, which are a result of the oxy- and deoxy-hemoglobin (Hb0₂, Hb), and water (H₂0). At the bottom of FIG. 6d, we list the recovered μₐ and μₛ′ values corresponding to the three selected wavelengths.

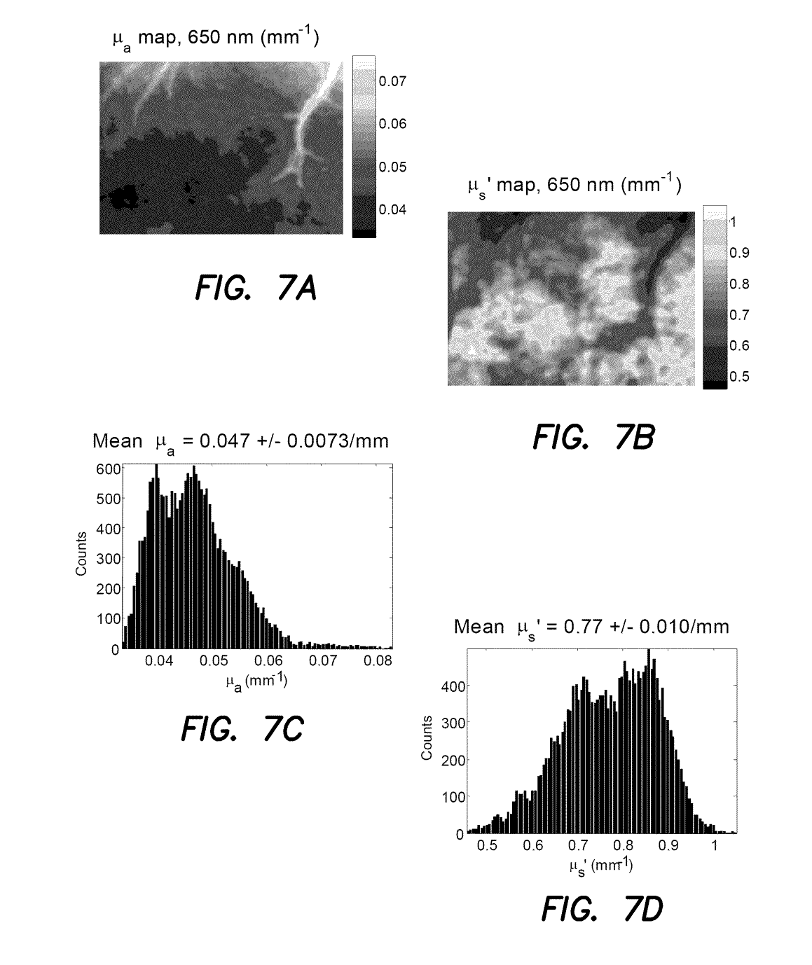

Pixel-by-pixel demodulation of spatial frequency data allows mapping of the absorption coefficients. In FIG. 7a we plot an example set of a μₐ map and in FIG. 7b a μₛ′ optical property map recovered at 650 nm. Note the strong absorption in the vein region, due to a strong absorption by Hb at this wavelength. In FIGS. 7c and 7d we show histogram distributions of the corresponding quantitative maps of FIGS. 7a and 7b, highlighting the spatial variation in recovered optical properties.

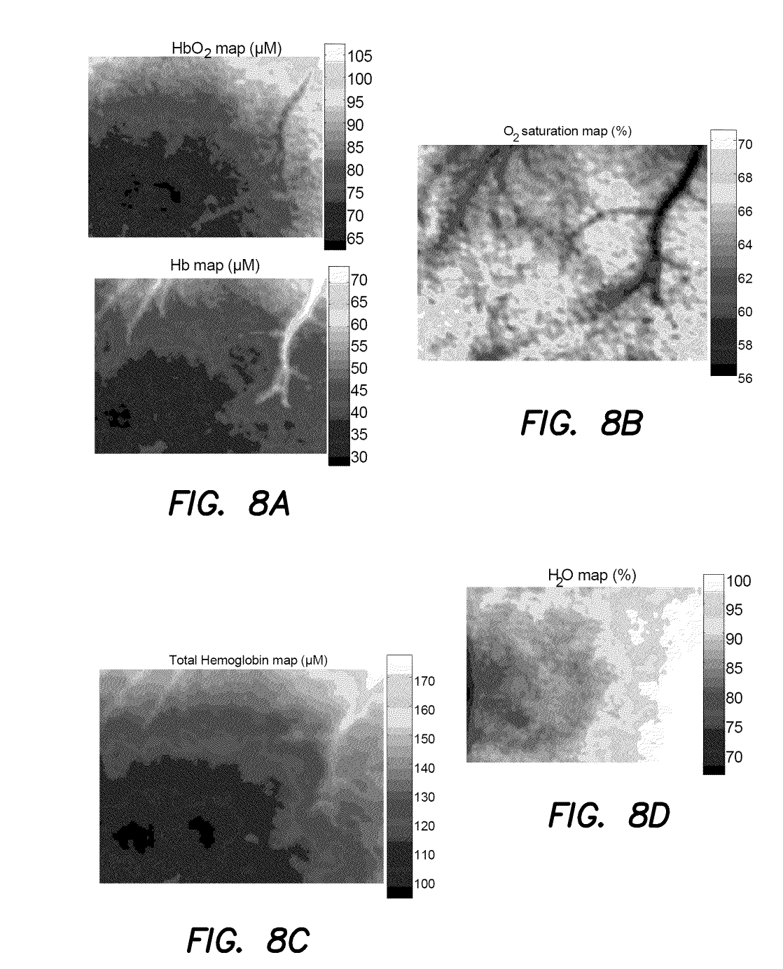

By mapping the absorption coefficient at each wavelength, we can perform quantitative spectroscopy of tissue. The result is a three dimensional data cube with an absorption spectrum at each spatial location. Knowledge of the extinction coefficients of the tissue chromophores (Hb0₂, Hb, and H₂0) allows us to fit these spectra to a linear Beer-Lambert absorption model. Consequently, we arrive at the quantitative concentrations of each chromophore, shown in FIG. 8a. Notice the low and high concentration of oxy and deoxy-hemoglobin, respectively, over the vein regions. This effect can be emphasized by calculating the tissue-level oxygen saturation (Sₜ0₂=Hb/[Hb+Hb0₂]), highlighting the effect of tissue oxygen extraction (FIG. 8b). Conversely, notice that the tissue regions are well perfused, with a high concentration of oxy-hemoglobin and Sₜ0₂ levels between 64 and 70%. The summation of Hb and Hb0₂ yields HbT, or the total hemoglobin concentration (FIG. 8c). Note that this quantitative, micromolar concentration is a direct, absolute measure of blood volume, a calculation unachievable with existing technologies. Lastly, if data is acquired at or near the water peak of 970 nm, tissue water concentration can also be measured. This direct measurement of tissue hydration, is depicted in FIG. 8d with units of percent concentration ranging from 75 to 100% of total volume.

Our long-term goal is to employ structured illumination to spatially resolve quantitative maps of tissue hemoglobin content and oxygen saturation in chronic wounds undergoing ischemia. We believe that the NIR-based structured illumination instrument can be used as a tissue oxygenation imager for quantitative assessment of hemoglobin content and oxygenation within ischemic superficial wounds. Furthermore, we expect the depth-sectioned imaging capability will enhance sensitivity to the physiologic changes in superficial wounds. The above disclosure of the illustrated embodiment of the invention establishes the feasibility of SI system as an effective tissue oxygenation imager in a pre-clinical animal wound model and to optimize measurement parameters necessary for developing a SI system for future human clinical trials and eventual diagnostic and therapeutic human use. The proposed animal skin flap model is known to undergo physiologic responses similar to chronic wounds with ischemia and provides a well-defined 2-layered tissue structures.

A cutaneous model for ischemic wounds is a random skin flap with a single pedicle. Pedicle flaps retain an existing blood supply. Random flaps refer to the skin flaps that lack specific connections to any blood vessels axial to the skin surface and are perfused by perforating vessels from the underlying wound bed. Two physiologic factors affect survival in random flaps, (1) blood supply to the flap through its base and (2) formation of new vascular channels between the flap and the underlying bed. In a single pedicle random flap, the pedicle or base of the flap is proximal to its blood supply and usually well perfused. The region of the flap furthest from the blood supply (the distal zone) is usually the region at highest risk of ischemia. This skin flap model is ideal for studying cutaneous ischemia because a gradient of blood perfusion is established along the length of the skin flap. In addition, re-attachment of the skin flap establishes a distinct two-layered wound model where the top layer is composed of both ischemia-induced necrotic region and healthy well-perfused region while the bottom layer is a healthy wound bed. A total of 20 rats weighing 300-400 grams have been studied. Results depicted in FIG. 1 illustrate multiwavelength absorption and reduced scattering properties of a typical in-vivo flap obtained 48 hrs post surgery.

Moving from the proximal to the distal zone of the flap, we observe 1) a steady increase in total hemoglobin (18-207 μM) and water fraction (28-85%), 2) a reduction in the oxygen saturation (78-25%), and 3) lowered reduced scattering in the distal (necrotic) region. These data demonstrate our ability to map superficial functional parameters using structured illumination. We intend to extend this technology to clinical studies for peripheral vascular disease, diabetic ulcers and decubitis ulcers in addition to burn triage and skin grafting and monitoring tissue response to photomodulation.

Consider another example where a swine model (Yorkshire White Pigs, 25-30 kg) was used to test the hypothesis that tissue spectroscopy using structured illumination can detect vascular occlusion in tissue transfer flaps. In order to test the SI device's ability to detect vascular occlusion, we created bilateral groin pedicled myocutaneous tissue transfer flaps based on the superficial and deep inferior epigastric vessels. Vascular occlusion of both the arterial and venous systems supplying the flaps were either completely occluded, or the flaps underwent selective venous occlusion. Measurements of the flaps were obtained using both structured illumination and digital color photography. Tissue chromophores measured using SI include oxygenated hemoglobin [HbO₂], deoxygenated hemoglobin [Hb], water fraction [H₂O %], and lipid content (fat %). Total hemoglobin [HbT] and Tissue Oxygen Saturation [SₜO₂] were then calculated based as previously discussed. Other endogenous chromophores (e.g. melanin, lipids (fat), other hemoglobins and heme breakdown products could also be measured.

Bilateral pedicled myocutaneous flaps were created based on the inferior epigastric vascular supply, with one side serving as the experimental side undergoing vascular occlusion, and the contralateral side serving as a control. We imaged both the control and experimental flaps simultaneously with SI prior to surgery, after the creation of the flaps, and during the experimental portion of the procedure during which vascular occlusion was performed. Baseline measurements were obtained after the surgical dissection of the flaps but prior to any occlusion of the flap's vasculature. Non-traumatic vascular clamps were then placed on the experimental side occluding both the superficial and deep inferior epigastric arteries and veins.

All six epigastric vessels (2 arteries and 4 veins) on the experimental flap were occluded using vascular clamps for 1 hour. During this period a set of time series measurements where acquired with the SI system. After 1 hour, the clamps were removed, allowing for reperfusion of the flap. After a period of re-equilibration, the flaps on the experimental side underwent complete selective suture ligation of the venous out-flow system, (100% venous occlusion). During this selective occlusion portion of the experiment, arterial inflow was not surgical obstructed, but allowed to continue to flow into the flap.

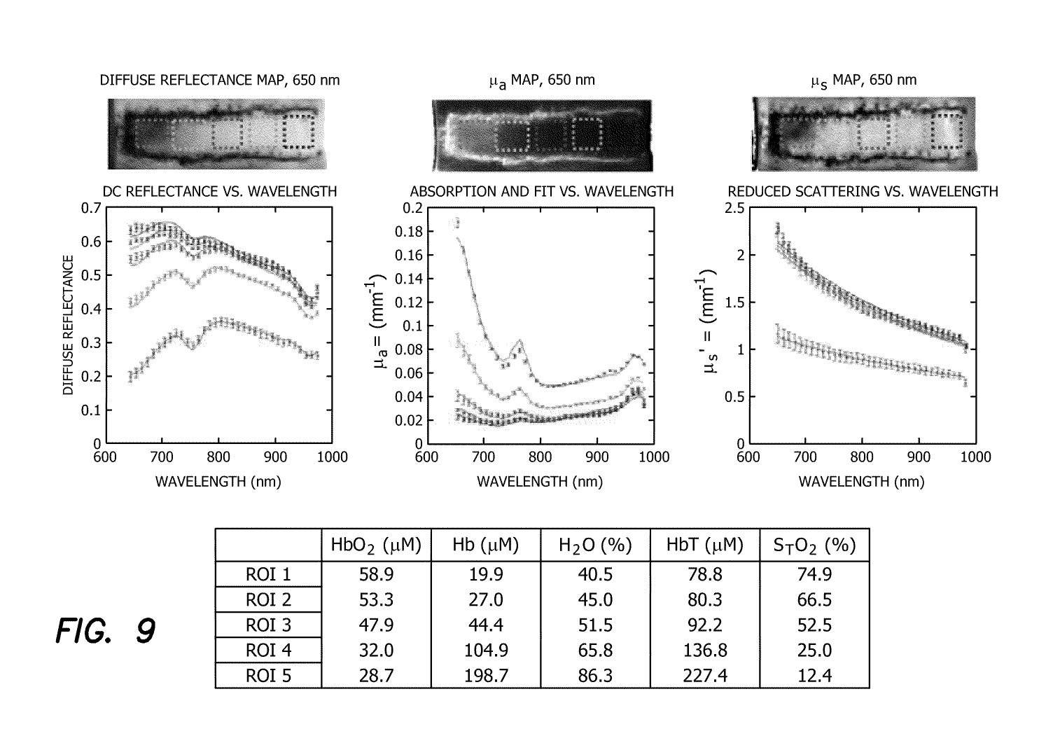

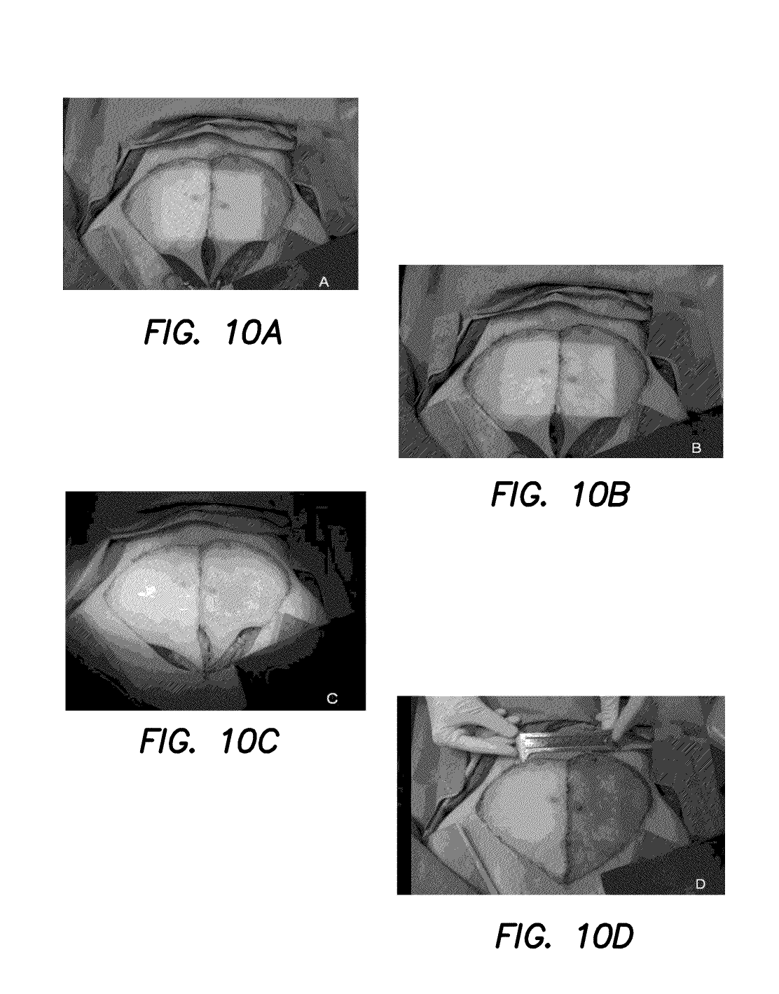

Color images shown in FIGS. 10a-10d, acquired using a consumer grade digital camera (Fuji Inc.), capture the clinical appearance of the flaps during arterial and venous occlusion at time=2 min (FIG. 10a), arterial and venous complete occlusion at time=60 min (FIG. 10b), selective venous occlusion at time=2 min (FIG. 10c), and selective venous occlusion at time=30 min (FIG. 10d). The dramatic changes to the flap undergoing complete venous obstruction are more obvious on visual inspection compared to combined arterial and venous obstruction.

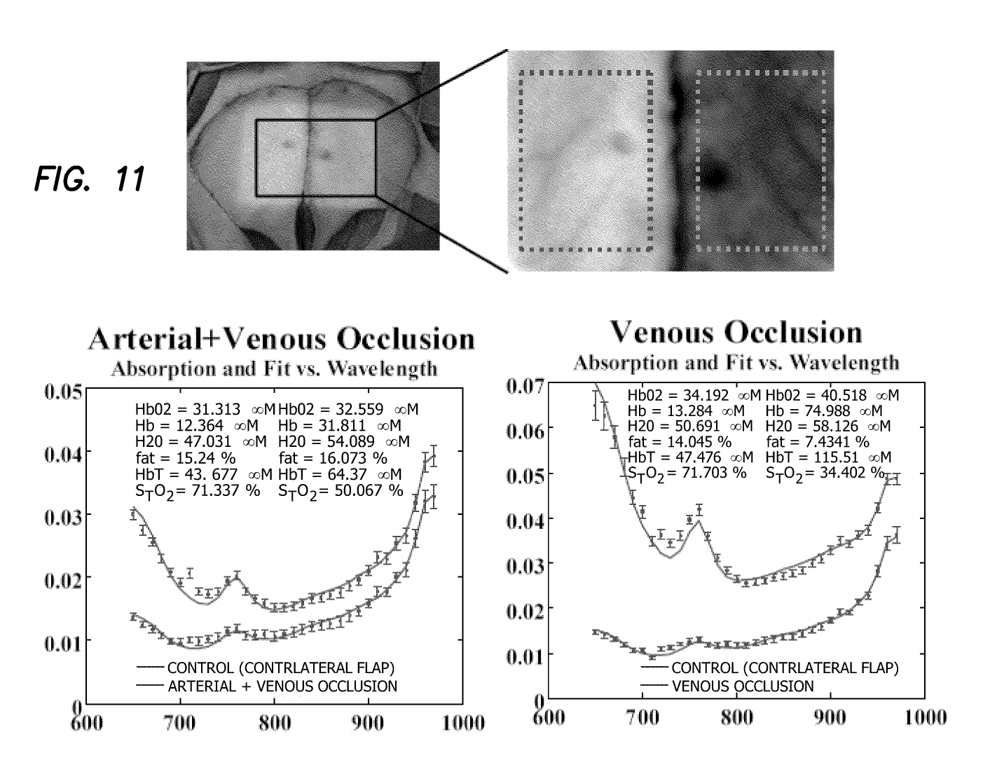

FIG. 11 illustrates SI results obtained for the complete venous occlusion and for combined arterial and venous occlusion. Using the diffuse reflectance image (650 nm, top right) we have defined a region of interest in which the spatially modulated light pattern is uniform. Data reduction on this region was performed as was done for the rat pedicle flap study. Optical properties were calculated for control and experiment subregions and chromophore concentrations were subsequently deduced from the wavelength-dependent absorption coefficient. Within 2 minutes of placement of the non-traumatic vascular clamps on the deep and superficial arteries and veins we observed that oxy hemoglobin concentration [HbO₂] was increased by 3.4% and deoxy hemoglobin concentration [Hb] had increased by 157.3%. Measured water fraction [H₂O %] and lipid concentration (fat %) demonstrate a slight increase by 15% and 5.4% respectively. Compared to control flap concentrations, the calculated total hemoglobin [HbT] increased by 47.4%, while tissue oxygen saturation [SₜO₂%] decreased by 29.8% in the occluded flap. Compiled results for venous occlusion and arterial and venous occlusion are presented in Table 1.

TABLE 1
Table 1- Comparison of Tissue Chromophores in both the Arterial
& Venous Ligation Flaps, and Selective Venous Occlusion Flaps.

| | Flap Type | | | | | |
|---|---|---|---|---|---|---|
| | Arterial & Venous Occlusion | | | Selective 100% Venous Occlusion | | |
| | | | % Δ from | | | % Δ from |
| | Control | Experimental | control flap | Control | Experimental | control flap |
| Chromophore | Flap | Flap | values at 2 min | Flap | Flap | values at 2 min |
| [HbO₂]μM | 31.313 | 32.559 | 4.0 | 34.192 | 40.518 | 18.5 |
| [Hb] μM | 12.364 | 31.811 | 157.3 | 13.284 | 74.988 | 464.5 |
| H₂O% | 47.031 | 54.089 | 15.0 | 50.691 | 58.126 | 14.7 |
| Fat % | 15.244 | 16.073 | 5.4 | 14.045 | 7.4341 | −47.1 |
| [HbT] μM | 43.677 | 64.37 | 47.4 | 47.476 | 115.51 | 143.3 |
| SₜO₂% | 71.337 | 50.067 | −29.8 | 71.703 | 34.402 | −52.0 |

Experimental flap values at 2 minutes post occlusion.

From the structured illumination measurements during the occlusion study, we found that all chromophores, except [H₂O %], changed to a greater extent in the selective venous occlusion flap compared to the combined arterial and venous occlusion flap. During selective venous occlusion both [HbO₂], and [HbT] increased to a greater extent than during the combined arterial and venous occlusion, 18.5% and 143.3% respectively. There was also a greater decrease in the calculated StO2 in the selective venous occlusion group (52%) compared to the combined arterial and venous occlusion flap (29.8%). Most notably the amount of [Hb] dramatically increased by 464.5% compared to the contralateral control flap, which was significantly larger than the change by 157.3% observed in the combined arterial and venous occlusion flap.

The data obtained from this set of initial experiments suggest that observable functional changes as reported by SI are quantitatively different depending on the occlusion mechanism. The large increase in [Hb] and [HbT] and corresponding decrease in [SₜO₂%] reflects the pooling of blood in the flap due to continued arterial inflow, which results in engorgement of the venous system with deoxygenated blood that is unable to exit the flap. These results agree with the more obvious changes seen visually in the venous obstruction portion of the experiment compared to the combined arterial and venous obstruction portion. Interestingly, the [SₜO₂%] measured following arterial and venous occlusion was marginally different from baseline. In this case we surmise that the ischemia and hypoxia to the flap resulted in vasodilatation at the capillary level, creating a “flushing” of oxygen-rich arterial blood to the bulk tissue and balancing the deoxygenation from the tissue's oxygen consumption. The fact that the same flap was used sequentially for both occlusion experiments may be a confounding variable. In future experiments we intend to only perform either arterial and venous occlusion or selective venous occlusion in experimental flap, and not both as in our initial experiment.

Each pig flap measurement presented here took approximately two minutes for acquisition. This allowed us to collect a large range of wavelengths (34) and spatial frequencies (4) to understand which data contained the optimal contrast for separating absorption, scattering, and chromophore data. In order to produce a clinically-viable Structured illumination instrument as proposed in Aim I, we have analyzed this information to identify a reduced data set that retained similar sensitivity, contrast, and resulting accuracy in chromophore estimation. First, in all skin data presented above, we have found that analysis of 2 or 3 spatial frequencies yield results within 10% of the full 4-frequency analysis. Secondly, we have determined that four well-chosen wavelengths yield similar accuracy for chromophore analysis, compared to the entire 34-wavelength data set. This has been confirmed using singular value decomposition (SVD) analysis to find wavelength sets that optimize chromophore value separation. We have identified a number of wavelength sets compatible with commercially-available high-power LEDs, including 690, 750, 830 and 980 nm, which provide accurate separation of chromophores Hb, HbO₂, and H₂O. Therefore, we have included within the scope of the illustrated embodiment a 2-spatial frequency, 4-light wavelength system which can be acquired in less than 1 second. In combination with online data processing capabilities this will enable immediate feedback on flap health status, reduce sensitivity to motion artifacts, and create the ability to track small, subtle changes that may occur during surgery. We therefore believe this modest change in hardware will be critical in order to allow physicians to identify perfusion changes deeper (up to 1 cm) and earlier than they can currently do via inspection.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A method of noncontact imaging for performing qualitative and quantitative analysis of wounds comprising performing structured illumination of surface and subsurface tissue by both diffuse optical tomography and rapid, wide-field quantitative mapping of tissue optical properties within a single measurement platform.

2. The method of claim 1 where performing structured illumination of surface and subsurface tissue comprises performing structured illumination to monitor a skin flap, a burn wound, a diabetic ulcer, a decubitis ulcer, a peripheral vascular disease, a skin graft, a bruise, and/or tissue response to photomodulation.

3. The method of claim 1 where performing structured illumination of surface and subsurface tissue comprises quantitative imaging of optical properties of superficial (0-5 mm depth) tissues in vivo.

4. The method of claim 1 where quantitative imaging of optical properties of superficial (0-5 mm depth) tissues in vivo comprises pixel-by-pixel demodulating and model based analysis of spatial frequency data to extract the local absorption and reduced scattering optical coefficients.

5. The method of claim 1 where performing structured illumination of surface and subsurface tissue further comprises multispectral imaging to separately analyze absorption spectra at each pixel to yield spatial maps of local oxy and deoxy hemoglobin concentration, and water concentration and to calculate total hemoglobin (THb) and oxygen saturation (SₜO₂) maps can then be calculated as THb=HHb+O₂Hb and SₜO₂=O₂Hb/[HHb+O₂Hb]*100, respectively.

6. A method of imaging comprising structured illumination of a cutaneous wound to spatially resolve quantitative maps of tissue hemoglobin, oxygenation and/or hydration in the wound.

7. The method of claim 6 where structured illumination of a cutaneous wound to spatially resolve quantitative maps of tissue hemoglobin content and oxygen saturation in the wound comprises structured illumination at various spatial frequencies can be processed to visualize depth-sectioned subsurface features in terms of scattering and absorption.

8. The method of claim 6 further comprising mapping the absorption coefficient at each wavelength in a predetermined spectral segment to perform quantitative spectroscopy of tissue.

9. The method of claim 8 where mapping the absorption coefficient at each wavelength in a predetermined spectral segment to perform quantitative spectroscopy of tissue comprises mapping extinction coefficients of the tissue chromophores.

10. The method of claim 9 where mapping extinction coefficients of the tissue chromophores comprises mapping concentration of oxy and deoxy-hemoglobin over the vein regions by calculating the tissue-level oxygen saturation (Sₜ0₂=Hb/[Hb+Hb0₂]), and highlighting the effect of tissue oxygen extraction.

11. The method of claim 9 where mapping extinction coefficients of the tissue chromophores comprises mapping a sum of Hb and Hb0₂ to yield HbT, the total hemoglobin concentration to obtain a direct, absolute measure of blood volume in tissue.

12. The method of claim 9 where mapping extinction coefficients of the tissue chromophores comprises mapping H₂O at or near the water peak of 970 nm to provide a direct mapping of tissue water concentration.

13. The method of claim 9 where mapping extinction coefficients of the tissue chromophores comprises mapping concentration of endogenous chromophores, including but not limited to melanin, lipids, hemoglobins and heme breakdown products.

14. The method of claim 6 where structured illumination of a cutaneous wound to spatially resolve quantitative maps of tissue hemoglobin, oxygenation and/or hydration in the wound comprises structured illumination to spatially resolve quantitative maps of tissue hemoglobin content and oxygen saturation in chronic wounds undergoing ischemia.

15. The method of claim 6 where structured illumination of a cutaneous wound to spatially resolve quantitative maps of tissue hemoglobin, oxygenation and/or hydration in the wound further comprises depth-sectioned imaging to enhance sensitivity to the physiologic changes in superficial wounds.

16. The method of claim 6 where structured illumination of a cutaneous wound to spatially resolve quantitative maps of tissue hemoglobin, oxygenation and/or hydration in the wound further comprises imaging using 690, 750, 830 and 980 nm light in a modulated pattern.

17. The method of claim 6 where structured illumination of a cutaneous wound to spatially resolve quantitative maps of tissue hemoglobin, oxygenation and/or hydration in the wound further comprises structured illumination of a cutaneous wound with online data processing to enable immediate feedback on flap health status, to reduce sensitivity to motion artifacts, to and create an ability to track small, subtle changes that may occur during surgery.

18. The method of claim 6 where structured illumination of a cutaneous wound to spatially resolve quantitative maps of tissue hemoglobin, oxygenation and/or hydration in the wound comprises identifying perfusion changes at tissue depths of 1 cm or less.

19. The method of claim 6 where structured illumination of a cutaneous wound to spatially resolve quantitative maps of tissue hemoglobin, oxygenation and/or hydration in the wound comprises performing the structured illumination with no more than two spatial frequencies to allow for rapid online data processing of an image.
